(19) United States
(12) Patent Application Publication
(10) Pub. No.: US 2023/0080175 A1
(43) Pub. Date: Mar. 16, 2023

(54) METHOD AND DEVICE FOR PREDICTING USER STATE

(71) Applicant: LOOXID LABS INC., Daejeon (KR)

(72) Inventors: Tae Heon LEE, Seoul (KR); Hong Gu LEE, Seoul (KR)

(73) Assignee: LOOXID LABS INC., Daejeon (KR)

(21) Appl. No.: 17/777,253

(22) PCT Filed: May 24, 2021
(86) PCT No.: PCT/KR2021/006429

§ 371 Date: May 16, 2022

(30) Foreign Application Priority Data
Jun. 3, 2020 (KR) 10-2020-0067095

(51) Int. Cl. G16H 50/20; G06N 5/02

(52) U.S. Cl. CPC G16H 50/20; G06N 5/022

(57) ABSTRACT

Provided are a method and device for predicting a user state according to an embodiment of the present invention. The method for predicting a user state according to an embodiment of the present invention comprises the steps of: acquiring first biometric data for a plurality of users; fine-tuning a prediction model on the basis of the first acquired biometric data and a fixed learning parameter; outputting a predicted user state using a fine-tuned prediction model by inputting a second biometric data for predicting the user state for predicting the user state for at least one user, wherein the fixed learning parameter is extracted on the basis of a first model that is different from the prediction model and trained to predict a user state for the plurality of users by inputting the first biometric data for the plurality of users.

### BACKGROUND OF THE DISCLOSURE

### Technical Field

The present invention relates to a method and device for predicting a user state.

### Background Art

Generally, biometric data such as electroencephalography (EEG) or electrocardiogram (ECG) is information expressing user's physical or psychological state. Biometric data is utilized in various fields such a medicine, psychology, or education.

Recently, in accordance with the development of artificial intelligence such as machine learning or deep learning, various analyzes have been attempted to understand biometric data using artificial intelligence technology.

However, unlike the image or voice data of the related art, it is difficult to collect biometric data. so that there is a problem in that an amount of data to be collected to be analyzed is small. Further, when any stimulus is applied to a user, there may be differences in biometric data to be collected depending on equipment to measure biometric data, an environment, and a user's state. Furthermore, when a large number of experienced specialists analyzes the collected biometric data, different analysis results can be obtained, thereby reducing the analysis accuracy.

Accordingly, a method for accurately predicting a user's state based on a small amount of collected biometric data is demanded.

### SUMMARY OF THE DISCLOSURE

An object to be achieved by the present invention is to provide a method and a device for predicting a user state.

Specifically, an object to be achieved by the present invention is to provide a method and a device for accurately predicting a user state based on a small amount of biometric data.

Objects of the present invention are not limited to the above-mentioned objects, and other objects, which are not mentioned above, may be clearly understood by those skilled in the art from the following descriptions.

In order to achieve the above-described object, provided are a method and device for predicting a user state according to an embodiment of the present invention.

A method for predicting a user state according to an embodiment of the present invention includes the steps of: acquiring a first biometric data for a plurality of users; fine-tuning a prediction model on the basis of the acquired first biometric data and a fixed learning parameter; and outputting a predicted user state using a fine-tuned prediction model by inputting a second biometric data for predicting the user state for predicting the user state for at least one user, in which the fixed learning parameter is extracted on the basis of a first model that is different from the prediction model and is trained to predict a user state for the plurality of users by inputting the first biometric data for the plurality of users.

A device for predicting a user state according to an embodiment of the present invention includes: a communication unit configured to transmit and receive data; and a control unit configured to be connected to the communication unit, in which the control unit is configured to acquire a first biometric data for a plurality of users through the communication unit, fine-tune a prediction model on the basis of the acquired first biometric data and a fixed learning parameter, and output a predicted user state using the fine-tuned prediction model by inputting a second biometric data for predicting a user state for at least one user, and the fixed learning parameter is extracted on the basis of a first model that is different from the prediction model and is trained to predict a user state for the plurality of users by inputting the first biometric data for the plurality of users.

Other detailed matters of the exemplary embodiments are included in the detailed description and the drawings.

According to the present invention, a learning parameter fixed by a pre-learning is used to accurately predict the user state using even a small amount of biometric data.

Further, according to the present invention, a prediction model with an improved performance more than the prediction model of the related art may be provided.

According to the present invention, the user state can be predicted by utilizing different biometric data at one time.

Further, according to the present invention, the analysis result of the trained parameter can be utilized to extract a signal pattern to be considered important in the bio signal.

The effects according to the present invention are not limited to the contents exemplified above, and more various effects are included in the present specification.

### BRIEF DESCRIPTION OF THE DRAWINGS

FIG. 1 is a schematic view for explaining a user state prediction system according to an embodiment of the present invention.

FIG. 2 is a schematic view of an electronic device according to an embodiment of the present invention.

FIG. 3 is an exemplary view for explaining a method for predicting a user state in an electronic device according to an embodiment of the present invention.

FIG. 4 is an exemplary view illustrating a signal shape or pattern corresponding to a trained parameter according to an embodiment of the present invention.

FIG. 5 is a flowchart for explaining a method for predicting a user state in an electronic device according to an embodiment of the present invention.

### DETAILED DESCRIPTION OF THE DISCLOSURE

Advantages and characteristics of the present invention and a method of achieving the advantages and characteristics will be clear by referring to embodiments described below in detail together with the accompanying drawings. However, the present invention is not limited to the following embodiments but may be implemented in various different forms. The embodiments are provided only to complete the disclosure of the present invention and to fully provide a person having ordinary skill in the art to which the present invention pertains with the category of the disclosure, and the present invention will be defined by the appended claims. In the description of drawings, like reference numerals denote like components.

In this specification, the terms “have”, “may have”, “include”, or “may include” represent the presence of the characteristic (for example, a numerical value, a function, an operation, or a component such as a part”), but do not exclude the presence of additional characteristic.

In the specification, the terms “A or B”, “at least one of A or/and B”, or “at least one or more of A or/and B” may include all possible combinations of enumerated items. For example, the terms “A or B”, “at least one of A or/and B”, or “at least one or more of A or/and B” may refer to an example which includes (1) at least one A, (2) at least one B, or (3) all at least one A and at least one B.

Although the terms “first”, “second”, and the like, may be used herein to describe various components regardless of an order and/or importance, the components are not limited by these terms. These terms are only used to distinguish one component from another. For example, a first user device and a second user device may refer to different user devices regardless of the order or the importance. For example, without departing from the scope of the present invention, a first component may be referred to as a second component, and similarly, a second component may be referred to as a first component.

When a component (for example, a first component) is referred to as being “operatively or communicatively coupled with/to” or “connected to” another component (for example, a second component), it can be understood that the component is directly connected to the other element or connected to the other element via another component (for example, a third component). In contrast, when a component (for example, a first component) is referred to as being “directly coupled with/to” or “connected to” another component (for example, a second component), it is understood that there may be another component (for example, a third component) between the components.

The terms “configured to (or set to)” may be exchangeably used with “suitable for”, “having the capacity to”, “designed to”, “adapted to”, “made to”, or “capable of” depending on the situation. The terms “configured to (or set)” may not necessarily mean only “specifically designed to” in a hardware manner. Instead, in some situation, the terms “a device configured to” may mean that the device “is capable of something” together with another device or components. For example, the terms “a processor configured (or set) to perform A, B, and C” may refer to a dedicated processor (for example, an embedded processor) configured to perform the corresponding operation or a generic-purpose processor (for example, a CPU or an application processor) which is capable of perform the operations by executing one or more software programs stored in a memory device.

The terms used in this specification are merely used to describe a specific embodiment, but do not intend to limit the scope of another embodiment. A singular form may include a plural form if there is no clearly opposite meaning in the context. Terms used herein including technical or scientific terms may have the same meaning as commonly understood by those skilled in the art. Among the terms used in this specification, terms defined in the general dictionary may be interpreted as having the same or similar meaning as the meaning in the context of the related art, but is not ideally or excessively interpreted to have formal meanings unless clearly defined in this specification. In some cases, even though the terms are defined in this specification, the terms are not interpreted to exclude the embodiments of the present specification.

The features of various embodiments of the present invention can be partially or entirely bonded to or combined with each other and can be interlocked and operated in technically various ways understood by those skilled in the art, and the embodiments can be carried out independently of or in association with each other.

“Biometric data” used in the present specification may be at least one of an electroencephalogram (EEC) signal and an electrocardiogram (ECG) signal which indicating a user's physical or psychological state. However, Biometric data is not limited thereto.

“Measuring device” used in the present specification may include all devices configured to acquire biometric data of the user. For example, the “measuring device” may be a head mounted display (HMD) device. In addition, the measuring device may be in contact with/worn on a part of the user's body, such as a headset, a smart ring, a smart watch, an ear set and/or an earphone. Further, the measuring device may include a device including a sensor for acquiring the user's biometric data and a content output device related to virtual reality, augmented reality or/and mixed reality. For example, when a HMD device includes a display unit, the measuring device may be the HMD device.

Hereinafter, various embodiments of the present invention will be described in detail with reference to accompanying drawings.

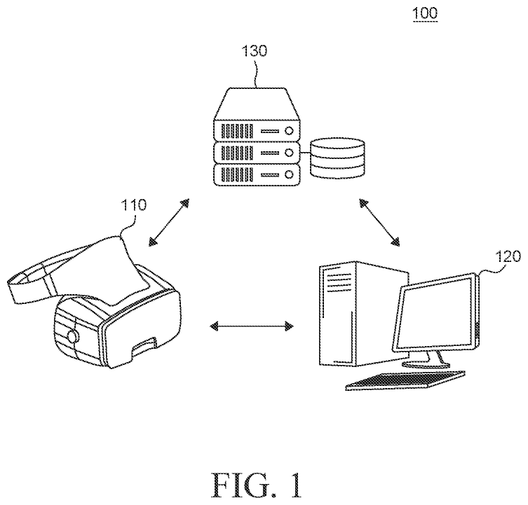

FIG. 1 is a schematic view for explaining a user state prediction system according to an embodiment of the present invention.

Referring to FIG. 1, the user state prediction system 100 is a system configured to predict a user state on the basis of a biometric data. The user state prediction system 100 includes a measuring device 110 configured to measure the biometric data of the user and an electronic device 120 configured to predict the user state on the basis of the biometric data. The user state prediction system 100 may further include a cloud server 130 configured to store the biometric data for each of a plurality of users.

First, the measuring device 110 is mounted on the user's head to provide multimedia content for virtual reality to the user so that the user can experience a spatial and temporal similarity to reality. At the same time, the measuring device 110 may be a complex virtual experience device capable of detecting physical, cognitive, and emotional changes of the user undergoing a virtual experience by acquiring the user's biometric data. For example, multimedia contents may include non-interactive images such as movies, animations, advertisements, or promotional images and interactive images interacting with the user, such as games, electronic manuals, electronic encyclopedias, or promotional images. However the multimedia contents are not limited thereto. Here, the image may be a three-dimensional image and include stereoscopic images.

The measuring device 110 may be a HMD device formed in structure that ca be worn on a head of the user's head. In this case, various multimedia contents for a virtual reality may be implemented in the form of processing inside the the HMD device. In addition, a content output device for providing multimedia contents is mounted on a part of the HMD device. And the mounted content output device may be implemented in such a way that the multimedia content is processed. For example, the multimedia contents may include contents for testing a cognitive ability, contents for measuring a health condition of the user, and/or contents for determining or diagnosing brain degenerative diseases such as dementia, Alzheimer's disease, or Parkinson's disease.

When the HMD device includes a display unit, one surface of the display unit may be disposed to be opposite to a face of the user so as to allow the user wearing the HMD device to check the multimedia contents.

According to various embodiments, an accommodation space for accommodating the content output device may be formed in a portion of the HMD device. When the content output device is accommodated in the accommodating space, the content output device may be disposed such that one surface (for example, one surface on the display unit of the content output device is located) of the content output device is opposite to the face of the user. For example, the content output device may include a portable a portable terminal device such as a smart phone or a tablet PC, or a portable monitor connected to a PC to output multimedia contents provided from the PC.

At least one sensor (not illustrated) for acquiring the user's biometric data may be formed at one side of the HMD device. For example, at least one sensor may include a brainwave sensor that measures at least one of the user's an electroencephalogram (EEC) signal and an electrocardiogram (ECG) signal.

According to various embodiments, at least one sensor is formed in a position to be contactable with the user's skin and when the user wears the HMD device, the sensor is in contact with the skin of the user to acquire the user's biometric data. In the present specification, it is described that the HMD device includes at least one sensor that acquires biometric data, but the present invention is not limited thereto. So that at least one sensor that acquires the user's biometric data may be mounted in the housing of the HMD device by means of a module separately from the HMD device. The expression of the HMD device is intended to include such a module or introduce the module itself.

The measuring device 110 may acquire the user's biometric data and may transmit the acquired biometric data to the electronic device 120 in accordance with the request of the electronic device 120. According to various embodiments, the measuring device 100 may transmit the measured biometric data to the cloud server 130. The biometric data transmitted by doing this may be stored in the cloud server 130.

The electronic device 120 may be connected to communicate with the measuring device 110. The electronic device 120 acquires the user's biometric data from the measuring device 110, and a personal computer (PC), a notebook, a workstation, or a smart TV for predicting the user's state based on the acquired biometric data. However, the electronic device 120 is not limited thereto. Here, the user state may include a sleep state, a health state, a cognitive state, an emotional state and/or a dementia progressing state, but is not limited thereto.

Specifically, the electronic device 120 acquires a first biometric data for a plurality of users from the measuring device 110 or the cloud server 130. The electronic device 120 may finely tune a prediction model for predicting the user state on the basis of the acquired the first biometric data and a fixed learning parameter. Here, the first biometric data is time-series biometric data. The first biometric data may be brainwave data for each of the plurality of users, but is not limited thereto.

The electronic device 120 for fine-tuning may extract the fixed learning parameter using a first model that is different from the above-mentioned prediction model. The first model is a model trained to predict the user state by inputting the first biometric data for the plurality of users. In other words, the electronic device 120 may train the first model to predict the user state by inputting the first biometric data. Here, the first model may include a plurality of layers and a second model having the same configuration as the above-described prediction model. Further, the plurality of layers may include a first layer for calculating feature data using similarity data representing a similarity between the first biometric data and a predetermined learning parameter used for the first model and a second layer for compressing the feature data.

The learning parameter used for the first model is updated by the above-described learning and when the learning is completed, the electronic device 120 may extract the updated learning parameter as a fixed learning parameter, from the first model. The electronic device 120 may perform the fine-tuning by applying the fixed learning parameter to the prediction model.

The electronic device 120 may output the predicted user state using a fine-tuned prediction model by inputting a second biometric data for predicting the user state for predicting the user state for at least one user. That is, the electronic device 120 may provide data representing the user state for at least one user.

In the meantime, the cloud server 130 may collect the biometric data for each of the plurality of users. The cloud server 130 may store the collected biometric data in association with each of the plurality of users. The cloud server 130 receives and stores the biometric data from the measuring device 110 or the electronic device 120. The cloud server 130 may transmit the biometric data to the electronic device 120 in accordance with the request of the electronic device 120.

As described above, according to the present invention, a learning parameter fixed by a pre-learning is used to accurately predict the user state using even a small amount of biometric data.

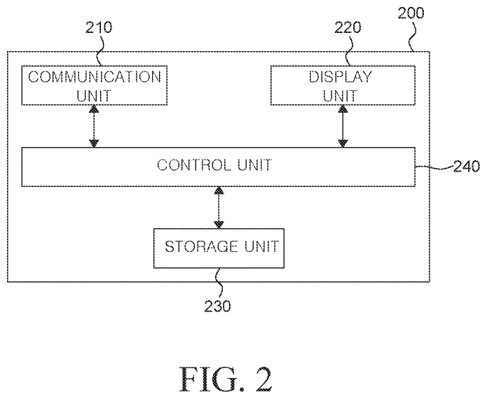

Hereinafter, the electronic device 120 will be described in detail with reference to FIG. 2.

FIG. 2 is a schematic view of an electronic device according to an embodiment of the present invention.

Referring to FIG. 2, the electronic device 200 includes a communication unit 210, a display unit 220, a storage unit 230, and a control unit 240. In the proposed embodiment, the electronic device 200 refers to the electronic device 120 of FIG. 1.

The communication unit 210 connects the electronic device 200 so as to communicate with the external device. The communication unit 210 is connected to the measuring device 110 using wired/wireless communication to transmit and receive various data. Specifically, the communication unit 210 may receive a first biometric data for the plurality of users and a second biometric data for predicting the user state, from the measuring device 110.

The display unit 220 may display various contents (for example, texts, images, videos, icons, banners, or symbols) to the user. For example, the display unit 220 may display an interface screen representing a predicted user state.

The storage unit 230 may store various data that is used to predict the user state on the basis the biometric data or may store various data generated through it.

According to various embodiments, the storage unit 230 may include at least one type of storage medium among flash memory type, hard disk type, multimedia card micro type, and card type memories (for example, SD or XD memory and the like), a random access memory (RAM), a static random access memory (SRAM), a read only memory (ROM), an electrically erasable programmable read only memory (EEPROM), a programmable read only memory (PROM), a magnetic memory, a magnetic disk, and an optical disk. The electronic device 200 may operate in association with a web storage that performs a storing function of the storage unit 230 on the Internet.

The control unit 240 is operatively connected to the communication unit 210, the display unit 220, and the storage unit 230. The control unit 240 may perform various commands to predict the user state on the basis of the biometric data.

Specifically, the control unit 240 acquires the first biometric data for a plurality of users from the measuring device 110 or the cloud server 130. The control unit 240 may finely tunes the prediction model on the basis of the acquired first biometric data and a fixed learning parameter. The control unit 240 may input the second biometric data for predicting the user state to the finely tuned prediction model, and the control unit 240 may output the predicted user state through the finely tuned prediction model. Here, the fixed learning parameter is different from the prediction model, and the fixed learning parameter may be extracted on the basis of the first model trained to predict the user state by inputting the first biometric data.

The process of fine-tuning the prediction model as described above will be described in detail below.

First, the control unit 240 may input the first biometric data for the plurality of users to the first model, so that the first model can be trained to predict the user state. when the training is completed, the control unit 240 may extract the fixed learning parameter of the first model. For example, the first model may include a plurality of layers for extracting a feature of the first biometric data and a second model with the same configuration as the prediction model. Here, even though the second model has the same configuration as the prediction model, the second model is different from the prediction model.

The plurality of layers may include layers that perform various operations for extracting feature from the first biometric data. The layers may include a first layer that calculates similarity data representing a similarity between the first biometric data and the predetermined learning parameter as feature data and a second layer that compresses the feature data. Here, the predetermined learning parameter may include a plurality of weights used to judge (or determine) the similarity in the first layer.

The control unit 240 may calculate the similarity data between the first biometric data and the predetermined learning parameter by means of the first layer. The control unit 240 may calculated feature data by performing a convolution on the calculated similarity data.

In order to calculate the similarity data, the control unit 240 may use cosine similarity, but is not limited thereto and various operations for calculating the similarity may be used. For example, the control unit 240 may convert the first biometric data and the learning data into one-dimensional vectors. The control unit 240 may calculate a cosine value between the one-dimensional vector of the first biometric data and the one-dimensional vector of the learning parameter as similarity data. In this case, a length of the learning parameter may be set to include a frequency band of the first biometric data.

The control unit 240 may perform the convolution operation for the cosine value calculated as described above. The control unit 240 may encode the result value into an activation vector to calculate the encoded result value as feature data. The control unit 240 may compress the feature data by means of the second layer to generate compressed data.

The control unit 240 may label or classify the user state using the second model trained to predict the user state by inputting the compressed data. In other words, the first model including the second model and the plurality of layers may be trained by the above-described operation.

According to various embodiments, the first layer is a cosine similarity based convolutional layer and the second layer is a max pooling layer. However, the first layer and the second layer are not limited thereto.

In order to calculate the feature data by means of the plurality of layers as described above, the control unit 240 may use the following Equation 1.

$$\frac{\partial E}{\partial {ω}_{i}^{k}} = \frac{E}{\partial {o}_{{j}^{*}}^{k}} \frac{{x}_{i + {j}^{*} - 1}}{\vert {X}_{[ {j}^{*} ; {j}^{*} + L - 1 ]} \vert}$$

Here, x refers to an input value of a neural network, w refers to a weighted vector of a convolutional filter, o refers to an output vector from the convolutional filter, W^{k} refers to a k-th filter vector of the convolutional layer, o^{k} refers to a k-th channel output vector from the convolutional layer, E refers to an output value of a neural network, L refers to a length of the convolutional filter, and i*, j* may refer to indexes of the convolution output vector having a maximum activation value.

When the learning parameter of the first model is updated by the above-described training, the control unit 240 may extract the updated learning parameter as a fixed learning parameter, from the first model. Here, the fixed learning parameter may refer to a parameter trained to predict the user state.

The control unit 240 may perform the fine-tuning by applying the fixed learning parameter to the prediction model. In other words, the control unit 240 may update the learning parameter updated in the first model including the second model and the plurality of layers as a parameter of layers constituting the prediction model.

The control unit 240 may acquire a second biometric data of at least one user from the measuring device 110. The control unit 240 may label or classify the user state using the fine-tuned prediction model by inputting the acquired second biometric data. For example, the fine-tuned prediction model may be multi-layered neural network classifier (MLP based classifier), but is not limited thereto.

As described above, according to the present invention, a learning parameter extracted by a pre-learning is applied to the prediction model to accurately predict the user state using a small amount of biometric data or different biometric data.

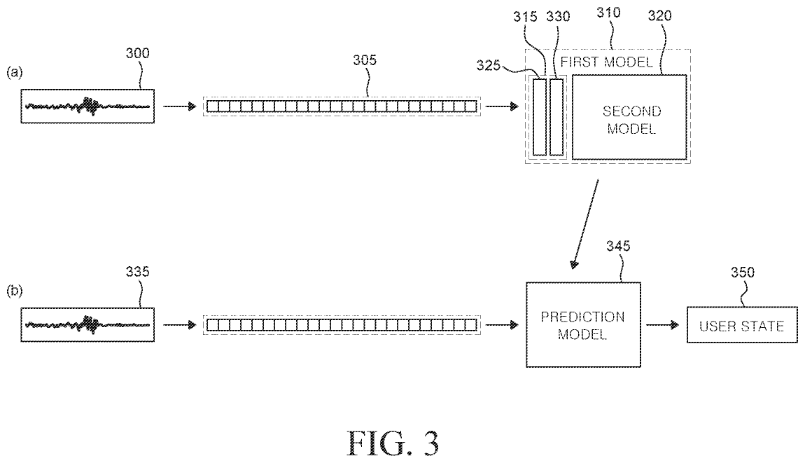

Hereinafter, a method for predicting a user state in the electronic device 120 will be described in more detailed with reference to FIGS. 1 and 3.

FIG. 3 is an exemplary view for explaining a method for predicting a user state in an electronic device according to an embodiment of the present invention.

Referring to FIGS. 1 and 3, the electronic device 120 may receive a first biometric data 300 for a plurality of uses used for learning, from the measuring device 110 or the cloud server 130 as illustrated in FIG. 3A. The electronic device 120 may convert the received first biometric data 300 into a one-dimensional vector 305. The biometric signal collected by the measuring device 110 may have a one-dimensional signal format for every channel at the time of collection. Here, the one-dimensional vector 305 may be a one-dimensional tensor that is a data format processible in a deep learning framework such as Pytorch or Tensorflow.

The electronic device 120 may train the first model 310 for predicting the user state by inputting the one-dimensional vector 305 as described above. Here, the first model 310 may include a plurality of layers 315 for extracting feature data from the biometric data and a second model 320 trained to predict the user state by inputting the output data output from the plurality of layers 315. For example, the plurality of layers 315 may include a cosine similarity based convolutional layer 325 and a max pooling layer 330. And the second model 320 may be a linear classifier for labeling or classifying which state of the user is indicated by the biometric signal, but is not limited thereto.

Among the plurality of layers 315, the cosine similarity based convolutional layer 325 may have a plurality of one-dimensional filters. By using such the plurality of one-dimensional filters 315, feature data corresponding to a significant signal shape or pattern can be extracted from the one-dimensional vector 305 of the biometric data. For example, in a sleep stage 2, sleep EEG signals corresponding to sleep spindles and K-complex appear, and the user's sleep state can be predicted using these EEG signals. The sleep spindles and the K-complex may be feature data corresponding to significant signal shape or pattern. According to various embodiments, in order to predict a cognitive state of the user, the brain wave signal may include feature data corresponding to a significant signal shape or pattern to determine the cognitive state of the user (for example, attention, language, spatiotemporal function, memory, and/or abstract thinking/executable).

In order to train to extract the feature data, the electronic device 120 may calculate a cosine similarity between the weights of the one-dimensional vector 305 and at least one filter of the cosine similarity based convolutional layer 325. And the electronic device 120 may perform the convolution on the calculated cosine similarity to calculate the feature data. Here, the calculated feature data may include a similarity between each piece of the one-dimensional vector 305 and each weight of each of the plurality of filters. Further, the cosine similarity based convolutional layer 325 may be allocated an output channel corresponding to the number of plurality of filters, and a cosine similarity value may becalculated t for every output channel. For example, when the cosine similarity based convolution layer 325 has 64 filters or 256 filters, 64 or 256 output channels may be allocated to each filter. In this case, the feature data may include a cosine similarity calculated for every output channel.

Among the plurality of layers 315, the max pooling layer 330 compresses the calculated feature data to output compressed data. In other words, the electronic device 120 may compress data so as to include a cosine similarity having the highest similarity among the cosine similarity calculated for every output channel by means of the max pooling layer 330 to output the compressed data. Here, when there are 64 output channels, the compressed data may be a vector configured by 64 values.

By this learning, the plurality of filters is updated by the learning to extract feature data such as a signal shape or pattern existing in the brainwave signal. so that when a vector corresponding to the compressed data is close to 1, it is determined that the input biometric data may have a signal shape or pattern which can be extracted by the plurality of filters.

In other words, the output compressed data may include information indicating whether there is a signal shape or pattern corresponding to the plurality of filters in the biometric data corresponding to the input value.

The electronic device 120 may output the labeled or classified user state using the second model 320 trained to label or classify the user state by inputting the compressed data. For example, when the input biometric data is sleep electroencephalogram data, the electronic device 120 may label or classify a user's sleep state among sleep states.

According to various embodiments, when there is a plurality of channels in the biometric data corresponding to an input value, the above-described training process may be repeated for every channel of the biometric data. The parameter of the first model 310 is also updated (that is, trained) by means of the learning.

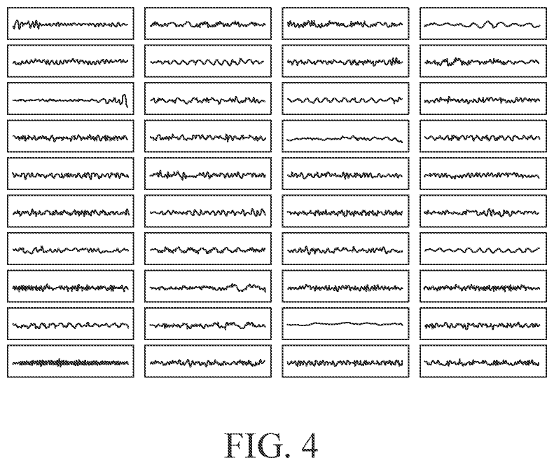

After the learning process, the electronic device 120 may extract a fixed learning parameter from the first model 310, and the electronic device 120 may apply the fixed learning parameter to the prediction model 345 to perform the fine-tuning. For example, the electronic device 120 may extract the plurality of updated filters of the cosine similarity based convolutional layer 325 as a fixed learning parameter. Or, the electronic device 120 may extract the updated filter of the cosine similarity based convolutional layer 325 and the updated filter of the second model 320 as fixed learning parameters, but is not limited thereto. The learning parameter updated by the learning process may be a parameter trained so as to correspond to the signal shape or pattern as illustrated in FIG. 4.

The electronic device 120 may apply the fixed learning parameter extracted as described above to layers located in the front stage among the plurality of layers constituting the prediction model 345, but is not limited thereto.

The electronic device 120 may receive a second biometric data 335 from the measuring device 110 or the cloud server 130, and the electronic device 120 may output the predicted user state 350 using the fine-tuned prediction model 345 by inputting the received second biometric data, as illustrated in FIG. 3B.

As described above, the previously trained parameter is applied to the prediction model so that the user state may be accurately predicted using a small amount of biometric data.

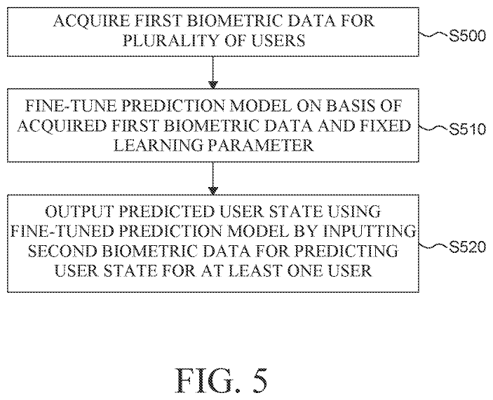

Hereinafter, a method for predicting a user state in the electronic device 120 will be described with reference to FIG. 5.

FIG. 5 is a flowchart for explaining a method for predicting a user state in an electronic device according to an embodiment of the present invention. Operations to be described below may be performed by a control unit 240 of the electronic device 200.

Referring to FIG. 5, the electronic device 200 acquires a first biometric data for a plurality of users (S500) and finely tunes the prediction model on the basis of the acquired first biometric data and a fixed learning parameter (S510).

Specifically, the electronic device 200 may receive the first biometric data for the plurality of users from the measuring device 110 or the cloud server 130. the electronic device 200 may train the first model to predict the user state by inputting the received first biometric data. For example, the first model includes a plurality of layers to extract feature data from the first biometric data and a second model which labels or classifies the user state by inputting the output data output from the plurality of layers. The learning parameter of the first model is also updated by the learning and the updated learning parameter may be extracted as a fixed learning parameter. The electronic device 200 may perform the fine-tuning by applying the fixed learning parameter to the prediction model.

The electronic device 200 outputs the predicted user state using the fine-tuned prediction model by inputting a second biometric data for predicting a user state for at least one user (S520).

By doing this, according to the present invention, a learning parameter fixed by a pre-learning is used to accurately predict the user state even using a small amount of biometric data.

The device and the method according to the embodiment of the present invention may be implemented as a program command which may be executed by various computers to be recorded in a computer readable medium. The computer readable medium may include solely a program command, a data file, and a data structure or a combination thereof.

The program commands recorded in the computer readable medium may be specifically designed or constructed for the present invention or those known to those skilled in the art of a computer software to be used. Examples of the computer readable recording medium include magnetic media such as a hard disk, a floppy disk, or a magnetic tape, optical media such as a CD-ROM or a DVD, magneto-optical media such as a floptical disk, and a hardware device which is specifically configured to store and execute the program command such as a ROM, a RAM, and a flash memory. Examples of the program command include not only a machine language code which is created by a compiler but also a high level language code which may be executed by a computer using an interpreter.

The above-described hardware device may operate as one or more software modules in order to perform the operation of the present invention and vice versa.

Although the embodiments of the present invention have been described in detail with reference to the accompanying drawings, the present invention is not limited thereto and may be embodied in many different forms without departing from the technical concept of the present invention. Therefore, the embodiments of the present invention are provided for illustrative purposes only but not intended to limit the technical concept of the present invention. The scope of the technical concept of the present invention is not limited thereto. Therefore, it should be understood that the above-described embodiments are illustrative in all aspects and do not limit the present invention. The protective scope of the present invention should be construed based on the following claims, and all the technical concepts in the equivalent scope thereof should be construed as falling within the scope of the present invention.

[National R&D Project which supports this invention]

### [Project Identification Number] 1711117604

### [Project Number] GK2000300

### [Department Name] Ministry of Science and ICT

[Project management (specialty) organization name] (Foundation) GigaKorea

### [Research Project] Integrated GigaKOREA Business(R & D)

[Research project name] 5G Based interactive immersive media technology development and demonstrate

### [Contribution Rate] 1/1

[Project execution organization] SK Broadband Co. Ltd.

### [Research Period] Jan. 1, 2020 to Dec. 31, 2020

What is claimed is:

1. A method for predicting a user state performed by a user state predicting apparatus, comprising the steps of:
acquiring a first biometric data for a plurality of users;
fine-tuning a prediction model on the basis of the acquired first biometric data and a fixed learning parameter; and
outputting a predicted user state using the fine-tuned prediction model by inputting a second biometric data for predicting the user state of at least one user,
wherein the fixed learning parameter is extracted on the basis of a first model that is different from the prediction model and is trained to predict a user state for the plurality of users by inputting the first biometric data for the plurality of users.

2. The method for predicting a user state of claim 1, wherein the step of fine-tuning includes the steps of:
extracting the fixed learning parameter using the first model by inputting the first biometric data; and
applying the fixed learning parameter to the prediction model.

3. The method for predicting a user state of claim 2, wherein the step of extracting the fixed learning parameter includes the steps of:
training the first model by inputting the first biometric data; and
extracting an updated learning parameter of the first model by the learning as the fixed learning parameter, from the first model.

4. The method for predicting a user state of claim 3, wherein the first model includes a plurality of layers and a second model having the same configuration as the prediction model.

5. The method for predicting a user state of claim 4, wherein the plurality of layers includes a first layer for calculating feature data using similarity data representing a similarity between the first biometric data and a predetermined learning parameter and a second layer for compressing the feature data.

6. The method for predicting a user state of claim 5, wherein the step of training the first model includes the steps of:
calculating the similarity data by means of the first layer;
calculating the feature data by performing convolution on the calculated similarity data;
compressing the calculated feature data by means of the second layer; and
labeling or classifying the user state for the plurality of users using the second model by inputting the compressed data.

7. The method for predicting a user state of claim 6, wherein the step of calculating the similarity data by means of the first layer includes the steps of:
converting the first biometric data and the learning parameter into a one-dimensional vector; and
calculating a cosine value between a one-dimensional vector of the first biometric data and a one-dimensional vector of the learning parameter as the similarity data.

8. The method for predicting a user state of claim 5, wherein the first layer is a cosine similarity based convolutional layer.

9. The method for predicting a user state of claim 5, wherein the second layer is a max pooling layer.

10. A device for predicting a user state, comprising:
a communication unit configured to transmit and receive data; and
a control unit configured to be connected to the communication unit,
wherein the control unit is configured to acquire a first biometric data for a plurality of users through the communication unit, fine-tune a prediction model on the basis of the acquired first biometric data and a fixed learning parameter, and
output a predicted user state using the fine-tuned prediction model by inputting a second biometric data for predicting a user state of at least one user, and
the fixed learning parameter is extracted on the basis of a first model that is different from the prediction model and is trained to predict a user state for the plurality of users by inputting the first biometric data for the plurality of users.

11. The device for predicting a user state of claim 10, wherein the control unit is configured to extract the fixed learning parameter using the first model by inputting the first biometric data and apply the fixed learning parameter to the prediction model.

12. The device for predicting a user state of claim 11, wherein the control unit is configured to train the first model by inputting the first biometric data and extract the updated learning parameter of the first model by the learning as the fixed learning parameter, from the first model.

13. The device for predicting a user state of claim 12, wherein the first model includes a plurality of layers and a second model having the same configuration as the prediction model.

14. The device for predicting a user state of claim 13, wherein the plurality of layers includes a first layer for calculating feature data using similarity data representing a similarity between the first biometric data and a predetermined learning parameter and a second layer for compressing the feature data.

15. The device for predicting a user state of claim 14, wherein the control unit is configured to calculate the similarity data by means of the first layer, calculate the feature data by performing convolution on the calculated similarity data, compress the calculated feature data by means of the second layer, and label or classify the user state for the plurality of users using the second model by inputting the compressed data.

16. The device for predicting a user state of claim 15, wherein the control unit is configured to convert the first biometric data and the learning parameter into a one-dimensional vector and calculate a cosine value between a one-dimensional vector of the first biometric data and a one-dimensional vector of the learning parameter as the similarity data.

17. The device for predicting a user state of claim 14, wherein the first layer is a cosine similarity based convolutional layer.

18. The device for predicting a user state of claim 14, wherein the second layer is a max pooling layer.
